(19) United States
(12) Patent Application Publication
(10) Pub. No.: US 2018/0206423 A1
(43) Pub. Date: Jul. 26, 2018

(54) MARINE BIOMASS REACTOR AND METHODS RELATED THERETO

(71) Applicant: SOUTHERN RESEARCH INSTITUTE, Birmingham, AL (US)

(72) Inventor: William Joseph Grieco, Vestavia, AL (US)

(21) Appl. No.: 15/878,738

(22) Filed: Jan. 24, 2018

Related U.S. Application Data
(60) Provisional application No. 62/449,747, filed on Jan. 24, 2017.

(51) Int. Cl. A01G 33/00; A01G 31/02; A01D 44/00

(52) U.S. Cl. CPC A01G 33/00; A01D 44/00; A01G 31/02

(57) ABSTRACT

Disclosed herein is a marine biomass reactor and methods related thereto that are capable of growing large quantities of a marine biomass material, such as, for example, macroalgae and/or aquatic macrophyte, at low cost.

### CROSS-REFERENCE TO RELATED APPLICATIONS

This Application claims the benefit of U.S. Provisional Application No. 62/449,747, filed on Jan. 24, 2017, which is incorporated herein by reference in its entirety.

### FIELD OF INVENTION

This invention relates to a marine biomass reactor for growing a marine biomass material. Also disclosed herein are methods of using a biomass reactor to grow and harvest a marine biomass material.

### BACKGROUND

It is desired to grow a marine biomass material, such as, for example, macroalgae and/or aquatic macrophyte, at large scale at low cost. Current technologies of growing and harvesting a marine biomass material are labor intensive and often results in low yields of the biomass material.

Accordingly, disclosed herein is a marine biomass reactor and methods related thereto that are capable of growing large quantities of a marine biomass material, such as, for example, macroalgae and/or aquatic macrophyte, at low cost.

### SUMMARY

Disclosed herein is a marine biomass reactor comprising: a) an outer shell having a central axis and a width and defining an inner volume, wherein the outer shell comprises: i) a water permeable outer shell material; ii) a first end cap and a second end cap spaced apart from the first end cap relative to the central axis, thereby defining a length of the outer shell; and iii) a spring coupled to the water permeable outer shell material having a first end and a second end spaced apart relative to the central axis, wherein the spring is configured to allow the outer shell to be compressed relative to the central axis, thereby decreasing the length of the outer shell; and b) a retraction line secured to the second end cap extending within the inner volume of the outer shell towards the first end cap.

Also disclosed herein is a biomass farm comprising two or more marine biomass reactors disclosed herein.

Also disclosed herein is a method of growing a marine biomass material comprising the steps of: a) loading a marine biomass material into the inner volume of a marine biomass reactor disclosed herein; and b) contacting the marine biomass material and at least a portion of the marine biomass reactor to water suitable for growing the marine biomass material, thereby growing the marine biomass material.

Also disclosed herein is a method of harvesting a marine biomass material comprising the steps of: a) securing the first end cap of a marine biomass reactor disclosed herein, wherein the inner volume of the marine biomass reactor comprises a marine biomass material; b) pulling the retraction line to compress the spring, thereby compressing the outer shell relative to the central axis, thereby decreasing the length of the outer shell; c) securing the compressed outer shell, thereby preventing the outer shell from expanding in length; and d) removing at least a portion of the marine biomass material from the inner volume of the marine biomass reactor.

### BRIEF DESCRIPTION OF THE FIGURES

These and other features of the preferred embodiments of the invention will become more apparent in the detailed description in which reference is made to the appended drawings wherein:

FIG. 1 shows an example of a marine biomass reactor disclosed herein in a deployed state.

FIG. 2 shows an example of a marine biomass reactor disclosed herein in an un-deployed state.

FIG. 3 shows an example of a marine biomass reactor disclosed herein in a deployed state during use.

FIG. 4 shows an example of a marine biomass farm having multiple marine biomass reactors.

FIG. 5 shows an example of a marine biomass reactor being seeded with a marine biomass material.

FIG. 6 shows an example of a marine biomass reactor disclosed herein in a deployed state during use containing a marine biomass material.

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DETAILED DESCRIPTION

The present invention can be understood more readily by reference to the following detailed description, examples, drawings, and claims, and their previous and following description. However, before the present articles, devices, systems, and/or methods are disclosed and described, it is to be understood that this invention is not limited to the specific devices, systems, and/or methods disclosed unless otherwise specified, as such can, of course, vary. It is also to be understood that the terminology used herein is for the purpose of describing particular aspects only and is not intended to be limiting.

The following description of the invention is provided as an enabling teaching of the invention in its best, currently known embodiment. To this end, those skilled in the relevant art will recognize and appreciate that many changes can be made to the various aspects of the invention described herein, while still obtaining the beneficial results of the present invention. It will also be apparent that some of the desired benefits of the present invention can be obtained by selecting some of the features of the present invention without utilizing other features. Accordingly, those who work in the art will recognize that many modifications and adaptations to the present invention are possible and can even be desirable in certain circumstances and are a part of the present invention. Thus, the following description is provided as illustrative of the principles of the present invention and not in limitation thereof.

A. Definitions

Various combinations of elements of this disclosure are encompassed by this invention, e.g. combinations of elements from dependent claims that depend upon the same independent claim.

Moreover, it is to be understood that unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; and the number or type of aspects described in the specification

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which this invention belongs. In this specification and in the claims which follow, reference will be made to a number of terms which shall be defined herein. As used throughout, the singular forms “a,” “an” and “the” include plural referents unless the context clearly dictates otherwise. Thus, for example, reference to “a marine biomass material” can include two or more marine biomass materials unless the context indicates otherwise.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, the terms “optional” or “optionally” mean that the subsequently described event or circumstance may or may not occur, and that the description includes instances where said event or circumstance occurs and instances where it does not.

The word “or” as used herein means any one member of a particular list and also includes any combination of members of that list.

B. Biomass Reactor and Methods

Disclosed herein is a marine biomass reactor comprising: a) an outer shell having a central axis and a width and defining an inner volume, wherein the outer shell comprises: i) a water permeable outer shell material; ii) a first end cap and a second end cap spaced apart from the first end cap relative to the central axis, thereby defining a length of the outer shell; and iii) a spring coupled to the water permeable outer shell material having a first end and a second end spaced apart relative to the central axis, wherein the spring is configured to allow the outer shell to be compressed relative to the central axis, thereby decreasing the length of the outer shell; and b) a retraction line secured to the second end cap extending within the inner volume of the outer shell towards the first end cap.

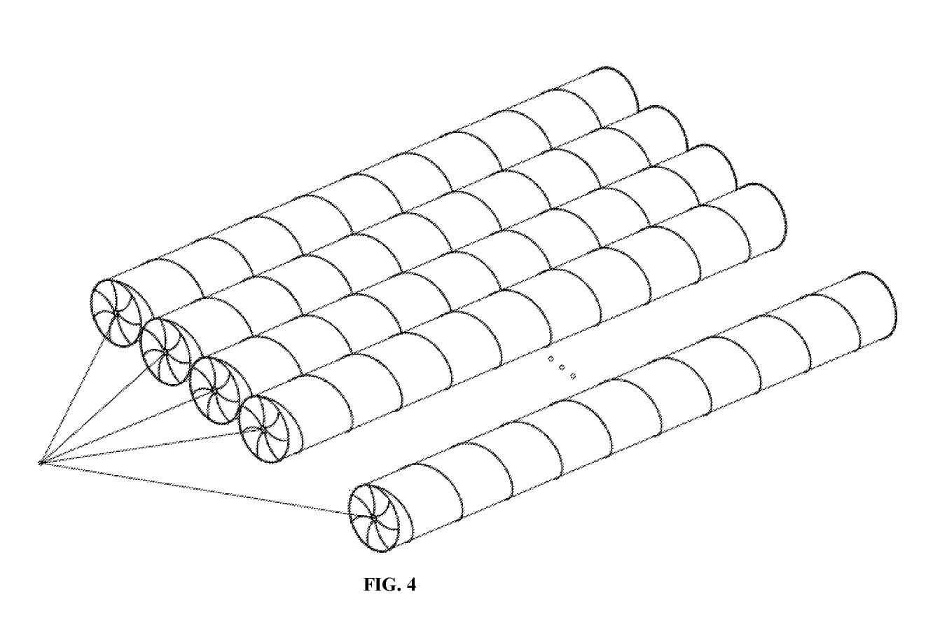

Also disclosed herein is a biomass farm comprising two or more marine biomass reactors disclosed herein. An example of biomass farm is shown in FIG. 4.

Also disclosed herein is a method of growing a marine biomass material comprising the steps of: a) loading a marine biomass material into the inner volume of a marine biomass reactor disclosed herein; and b) contacting the marine biomass material and at least a portion of the marine biomass reactor to water suitable for growing the marine biomass material, thereby growing the marine biomass material.

Also disclosed herein is a method of harvesting a marine biomass material comprising the steps of: a) securing the first end cap of a marine biomass reactor disclosed herein, wherein the inner volume of the marine biomass reactor comprises a marine biomass material; b) pulling the retraction line to compress the spring, thereby compressing the outer shell relative to the central axis, thereby decreasing the length of the outer shell; c) securing the compressed outer shell, thereby preventing the outer shell from expanding in length; and d) removing at least a portion of the marine biomass material from the inner volume of the marine biomass reactor.

The marine biomass reactor disclosed herein allows for large quantities of a marine biomass material to be grown and harvested at low cost. The marine biomass reactor can be used in water suitable for growing the marine biomass material. An amount of marine biomass material can be placed into the inner volume of the marine biomass reactor. The marine biomass material will grow when the marine biomass reactor is placed in water suitable for growing the marine biomass material. In one aspect, the water suitable for growing the marine biomass material is rich in nutrients that promote the growth of marine biomass material. For example, the water suitable for growing the marine biomass material can be ocean water. In another example, the water suitable for growing the marine biomass material can be brackish water. In yet another aspect, the water suitable for growing the marine biomass material can be fresh water. An increase in nutrients that promote the growth of marine biomass material can be found in proximity to, for example, deltas, run offs from agriculture, and fish farms.

The water permeable outer shell material allows water to enter the inner volume of the marine biomass reactor and come into contact with the marine biomass material. The water permeable outer shell material can also be substantially marine biomass material impermeable, thereby containing marine biomass material within the marine biomass reactor, and preventing marine biomass material from exiting thru the water permeable outer shell material.

In one aspect, the water permeable outer shell material is a mesh. In one aspect, the mesh is configured to allow water to pass thru the mesh, but substantially prevent marine biomass material from passing thru the mesh. In another aspect, the water permeable outer shell material can comprise a woven fabric or a non-woven fabric, or a combination thereof. In one aspect, the water permeable outer shell material can be a synthetic or natural netting.

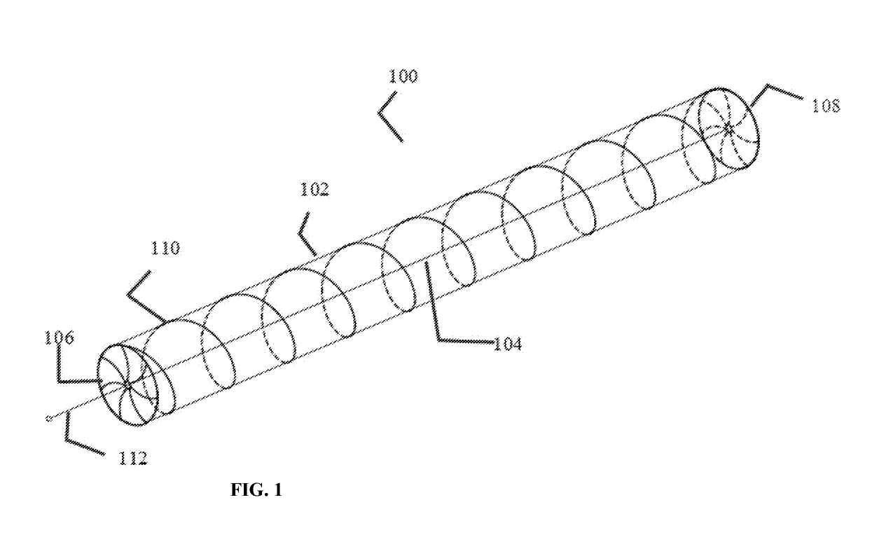

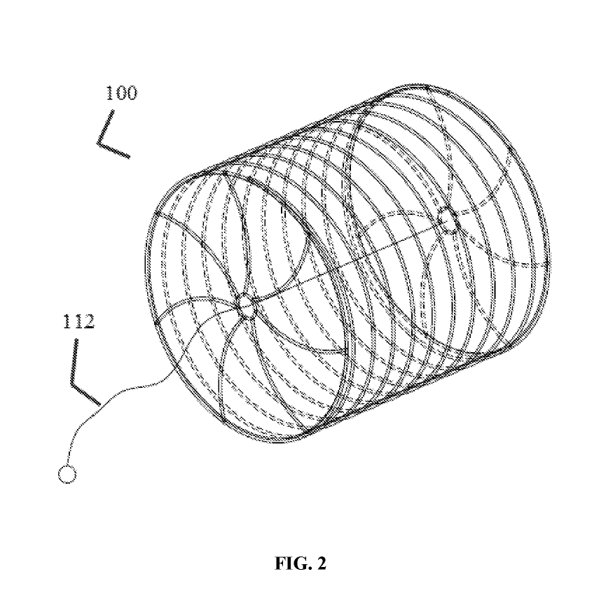

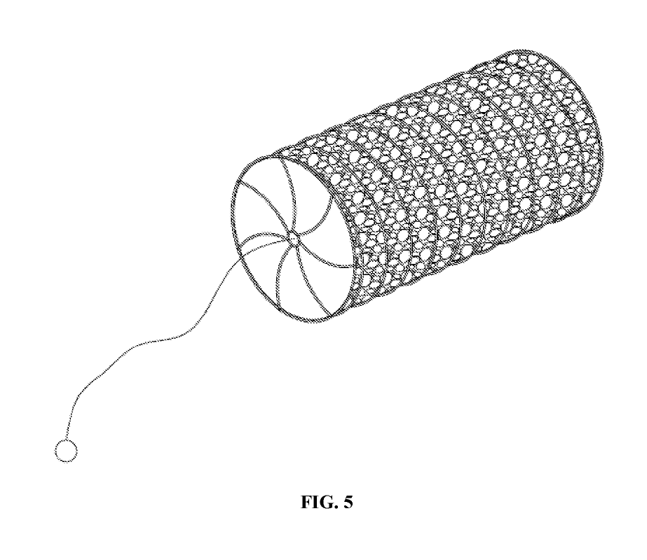

The marine biomass reactor can be in a deployed state where the spring is allowed to expand to give the marine biomass reactor a length that is used during the growth of the marine biomass material, see FIGS. 1, 3, and 6. The marine biomass reactor can be in an un-deployed state where the spring is compressed to shorten the length of the marine biomass reactor as compared to deployed state, see FIGS. 2 and 5. The length of the marine biomass reactor in an un-deployed state can be locked in via a locking mechanism that prevents the spring from expanding. The un-deployed state is used when the marine biomass reactor is stored to minimize the space occupied by the marine biomass reactor. Also, the un-deployed state can be used when the marine biomass material is loaded into the marine biomass reactor. Also, the un-deployed state can be used when the marine biomass material is harvested from the marine biomass reactor. Shortening the length of the marine biomass reactor will compress and make the marine biomass material more dense, which will make it easier to remove from the marine biomass reactor.

In one aspect, the inner volume of the outer shell can comprise at least 0.001 vol % of the marine biomass material. For example, the inner volume of the outer shell can comprise at least 0.01 vol %, 0.05 vol %, 0.1 vol %, 0.5 vol %, or 1.0 vol % of the marine biomass material. For example, the inner volume of the outer shell can comprise from 0.001 vol % to 5.0 vol % of the marine biomass material, such as from 0.01 vol % to 1.0 vol % of the marine biomass material.

In one aspect, the wherein the spring is a wound spring. In another aspect, the spring extends from the first end cap to the second end cap. In one aspect, the spring can be a metal spring or a polymer spring. The retraction line is secured to the second end cap. The length of the retraction line is such that the retraction line can be accessed from the first end cap, or when the first end cap is removed. In one aspect, the first end cap comprises an opening configured to receive the retraction line. The outer shell is secured from first end cap or other part of the outer shell if the first end cap is detached and removed. The retraction line is pulled in a direction towards the first end cap or previous location of the first end cap. This will compress the spring, thereby compressing the outer shell relative to the central axis, thereby decreasing the length of the outer shell. Thus, the second end cap is moved in a direction towards the first end cap relative to the central axis when the retraction lined is pulled. At a desired length, the retraction line and/or the outer shell is secured to prevent the spring from expanding. Marine biomass material can then be harvested or seeded to the inner volume of the marine biomass reactor.

In one aspect, the second end cap is made of a material suitable to secure the retraction line and to withstand the force applied when the retraction line is pulled to compress the spring. For example, the second end cap can comprise a metal. In another aspect, the second end cap can comprise a water permeable material.

In one aspect, the first end cap is made of a material suitable to secure the outer shell when the retraction line is pulled to compress the spring. For example, the first end cap can comprise a metal. In another aspect, the first end cap can comprise a water permeable material.

In one aspect, the outer shell can have a cross-sectional shape selected from the group consisting of circular, rectangular, triangular, oval, square, polygonal, pentagonal, hexagonal, heptagonal, and octagonal.

In one aspect, the width of outer shell can be at least 50 centimeter, 1 meter, 3 meters, 5 meters, or 10 meters. For example, the width of outer shell can be from 50 centimeter to 20 meters.

In another aspect, the length of outer shell can be at least 5 meters, 10 meters, 25 meters, 50 meters, 75 meters, 100 meters, 250 meters, 500 meters, or 750 meters. For example, the length of outer shell can be from 5 meters to 1,000 meters.

In one aspect, only a portion of the marine biomass material is removed from the marine biomass reactor during harvesting. A portion of the marine biomass material can be left in the marine biomass reactor to function as the seed marine biomass material to continue the growth of the marine biomass material when the marine biomass reactor is again deployed in water suitable for growing the marine biomass material to grow more marine biomass material.

A marine biomass material can be loaded within the inner volume of the outer shell when the marine biomass reactor is in a deployed or un-deployed state.

In one aspect, the marine biomass material is allowed to move freely within the inner volume of the marine biomass reactor.

In one aspect, the marine biomass reactor can further comprise a buoyancy device secured to the outer shell, retraction line, or both the outer shell and the retraction line. For example, the marine biomass reactor can further comprise one or more buoyancy devices secured to the outer shell, retraction line, or both the outer shell and the retraction line. The buoyancy device secured prevents the marine biomass reactor from sinking. The buoyancy device can also be used to locate a biomass reactor that is allowed to float freely in a body of water. In one aspect, the buoyancy device can be a buoy.

In one aspect, the biomass reactor with or without a buoyancy device is configured to allow at least 10%, 20%, 30%, 40%, or 50% of the width of the marine biomass reactor to be above water during use. For example, the biomass reactor without a buoyancy device is configured to allow at least 10%, 20%, 30%, 40%, or 50% of the width of the marine biomass reactor to be above water during use. In another example, biomass reactor with a buoyancy device or one or more buoyancy devices is/are configured to allow at least 10%, 20%, 30%, 40%, or 50% of the width of the marine biomass reactor to be above water during use.

In one aspect, the outer shell and/or the retraction line can further be secured to an anchor. The anchor secures the marine biomass reactor to a specific location, thereby preventing the marine biomass reactor from being moved by currents, waves, or wind.

In one aspect, the marine biomass material comprises macroalgae. For example, the macroalgae can be selected from the group consisting of ulva, garcilaria, and saragassum, or a combination thereof.

In another aspect, the marine biomass material comprises an aquatic macrophyte. For example, the aquatic macrophyte is selected from the group consisting of genus Azolla, Lemna, Myriophyllum, Salvinia, Spirodela, and Wolffia, or a combination thereof

In another example, the aquatic macrophyte can be selected from the group consisting of the species Azolla caroliniana, Azolla circinata, Azolla filiculoides, Azolla japonica, Azolla mexicana, Azolla microphylla, Azolla nilotica, Azolla pinnata, Azolla rubra, Lemna aequinoctialis, Lemna perpusilla, Lemna minor, Lemna gibba, Lemna trisulca, Lemna minuta, Lemna valdiviana, Myriophyllum alpinum, Myriophyllum alterniflorum DC., 1815, Myriophyllum amphibium, Myriophyllum aquaticum, Myriophyllum artesium, Myriophyllum austropygmaeum, Myriophyllum axilliflorum, Myriophyllum balladoniense, Myriophyllum bonii, Myriophyllum callitrichoides, Myriophyllum caput-medusae, Myriophyllum coronatum, Myriophyllum costatum, Myriophyllum crispatum, Myriophyllum dicoccum, Myriophyllum drummondii, Myriophyllum decussatum, Myriophyllum echinatum, Myriophyllum exasperatum, Myriophyllum farwellii, Myriophyllum filiforme, Myriophyllum glomeratum, Myriophyllum gracile, Myriophyllum heterophyllum, Myriophyllum hippuroides, Myriophyllum humile, Myriophyllum implicatum, Myriophyllum indicum Willd., 1805, Myriophyllum integrifolium, Myriophyllum jacobsii, Myriophyllum lapidicola, Myriophyllum latifolium, Myriophyllum laxum, Myriophyllum limnophilum, Myriophyllum lophatum, Myriophyllum mattogrossense, Myriophyllum mezianum, Myriophyllum muelleri, Myriophyllum muricatum, Myriophyllum oguraense, Myriophyllum oliganthum, Myriophyllum petraeum, Myriophyllum papillosum, Myriophyllum pedunculatum, Myriophyllum pinnatum, Myriophyllum porcatum, Myriophyllum propinquum, Myriophyllum pygmaeum, Myriophyllum quitense, Myriophyllum robustum, Myriophyllum salsugineum, Myriophyllum siamense, Myriophyllum sibiricum, Myriophyllum simulans, Myriophyllum spicatum, Myriophyllum striatum, Myriophyllum tenellum, Myriophyllum tetrandrum, Myriophyllum trachycarpum, Myriophyllum trifidum, Myriophyllum triphyllum, Myriophyllum tuberculatum, Myriophyllum ussuriense, Myriophyllum variifolium, Myriophyllum verrucosum, Myriophyllum verticillatum, Myriophyllum votschii, Salvinia auriculata, Salvinia biloba, Salvinia cucullata, Salvinia hastata, Salvinia herzogii, Salvinia minima, Salvinia molesta, Salvinia natans, Salvinia nymphellula, Salvinia oblongifolia, Salvinia radula, Salvinia sprucei, Spirodela intermedia, Spirodela oligorrhiza, Spirodela polyrrhiza, Spirodela punctata, Spirodela sichuanensis, Wolffia angusta, Wolffia arrhiza, Wolffia australiana, Wolffia borealis, Wolffia brasiliensis, Wolffia columbiana, Wolffia cylindracea, Wolffia elongata, Wolffia globosa, Wolffia microscopica, and Wolffia neglecta, or a combination thereof.

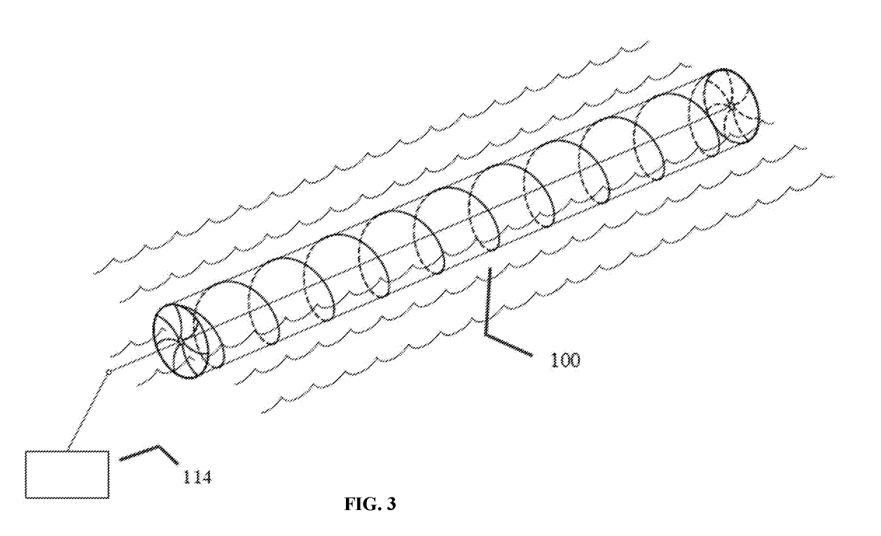

FIGS. 1-6 show a bioreactor 100. The bioreactor 100 has an outer shell 102. The outer shell 102 has a central axis 104. The outer shell 102 defines an inner volume of the bioreactor 100, when the bioreactor is in a deployed state, as shown in FIG. 1. The outer shell 102 also has a width. The outer shell 102 comprises a water permeable outer shell material. The outer shell 102 also comprises a first end cap 106 and a second end cap 108 that are spaced apart relative to the central axis 104, thereby defining a length of the outer shell 102. The outer shell 102 also comprises a spring 110 coupled to the water permeable outer shell material having a first end and a second end spaced apart relative to the central axis, wherein the spring 110 is configured to allow the outer shell 102 to be compressed relative to the central axis 104, thereby decreasing the length of the outer shell 102. The compressed state of the biomass reactor 100 is shown in FIG. 2. The biomass reactor 100 also comprises a retraction line 112 that is secured to the second end cap 108 extending within the inner volume of the outer shell towards the first end cap 106. The retraction line 112 can be received in an opening in the first end cap 106. The first end cap 106 can be secured such that the retraction line 112 can be pull the second end cap 10 towards the first end cap 106, thereby decreasing the length of the bioreactor 100. This also concentrates any biomass material present in the biomass reactor 100, which makes it easier to harvest the biomass from the biomass reactor 100. As shown in FIG. 3, the retraction line 112 can be attached to an anchor 114 that is configure to keep the biomass reactor 100 stationary during use. The anchor 114 can alternatively be attached to the outer shell 102 instead of or in addition to the retraction line 112.

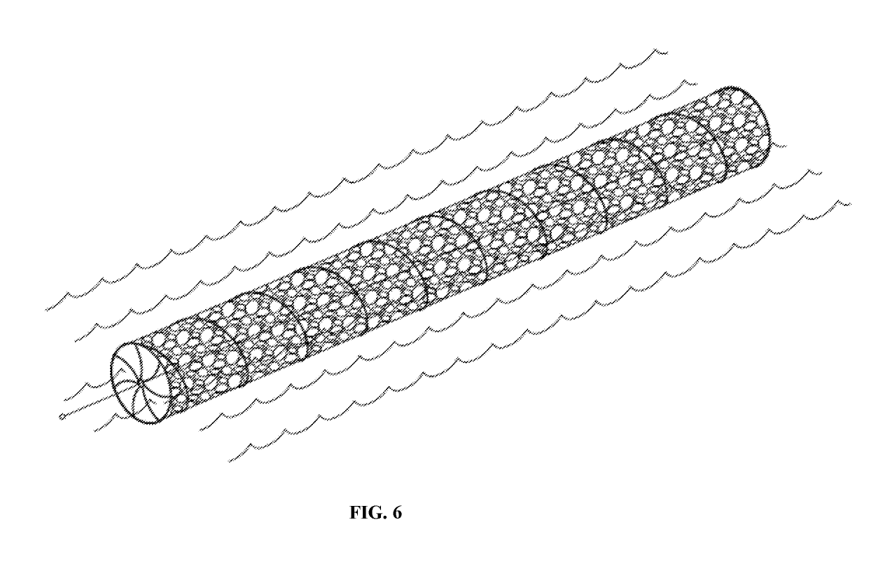

FIG. 5 is a replica of FIG. 2 but also shows biomass being trapped inside on the bioreactor. FIG. 6 shows a bioreactor in a deployed state with biomass being trapped inside on the bioreactor.

C. Aspects

In view of the described marine biomass reactor and methods and variations thereof, herein below are described certain more particularly described aspects of the inventions. These particularly recited aspects should not however be interpreted to have any limiting effect on any different claims containing different or more general teachings described herein, or that the “particular” aspects are somehow limited in some way other than the inherent meanings of the language and formulas literally used therein.

Aspect 1: A marine biomass reactor comprising: a) an outer shell having a central axis and a width and defining an inner volume, wherein the outer shell comprises: i. a water permeable outer shell material; ii. a first end cap and a second end cap spaced apart from the first end cap relative to the central axis, thereby defining a length of the outer shell; and iii. a spring coupled to the water permeable outer shell material having a first end and a second end spaced apart relative to the central axis, wherein the spring is configured to allow the outer shell to be compressed relative to the central axis, thereby decreasing the length of the outer shell; b) a retraction line secured to the second end cap extending within the inner volume of the outer shell towards the first end cap.

Aspect 2: The marine biomass reactor of aspect 1, wherein the biomass reactor further comprises a marine biomass material within the inner volume of the outer shell.

Aspect 3: The marine biomass reactor of aspect 2, wherein the marine biomass material comprises macroalgae.

Aspect 4: The marine biomass reactor of aspect 3, wherein the macroalgae is selected from the group consisting of ulva, garcilaria, and saragassum, or a combination thereof.

Aspect 5: The marine biomass reactor of aspect 2, wherein the marine biomass material comprises an aquatic macrophyte.

Aspect 6: The marine biomass reactor of aspect 5, wherein the aquatic macrophyte is selected from the group consisting of the genus Azolla, Lemna, Myriophyllum, Salvinia, Spirodela, and Wolffia, or a combination thereof.

Aspect 7: The marine biomass reactor of aspect 5, wherein the aquatic macrophyte is selected from the group consisting of the species Azolla caroliniana, Azolla circinata, Azolla filiculoides, Azolla japonica, Azolla mexicana, Azolla microphylla, Azolla nilotica, Azolla pinnata, Azolla rubra, Lemna aequinoctialis, Lemna perpusilla, Lemna minor, Lemna gibba, Lemna trisulca, Lemna minuta, Lemna valdiviana, Myriophyllum alpinum, Myriophyllum alterniflorum DC., Myriophyllum amphibium, Myriophyllum aquaticum, Myriophyllum artesium, Myriophyllum austropygmaeum, Myriophyllum axilliflorum, Myriophyllum balladoniense, Myriophyllum bonii, Myriophyllum callitrichoides, Myriophyllum caput-medusae, Myriophyllum coronatum, Myriophyllum costatum, Myriophyllum crispatum, Myriophyllum dicoccum, Myriophyllum drummondii, Myriophyllum decussatum, Myriophyllum echinatum, Myriophyllum exasperatum, Myriophyllum farwellii, Myriophyllum filiforme, Myriophyllum glomeratum, Myriophyllum gracile, Myriophyllum heterophyllum, Myriophyllum hippuroides, Myriophyllum humile, Myriophyllum implicatum, Myriophyllum indicum Willd., Myriophyllum integrifolium, Myriophyllum jacobsii, Myriophyllum lapidicola, Myriophyllum latifolium, Myriophyllum laxum, Myriophyllum limnophilum, Myriophyllum lophatum, Myriophyllum mattogrossense, Myriophyllum mezianum, Myriophyllum muelleri, Myriophyllum muricatum, Myriophyllum oguraense, Myriophyllum oliganthum, Myriophyllum petraeum, Myriophyllum papillosum, Myriophyllum pedunculatum, Myriophyllum pinnatum, Myriophyllum porcatum, Myriophyllum propinquum, Myriophyllum pygmaeum, Myriophyllum quitense, Myriophyllum robustum, Myriophyllum salsugineum, Myriophyllum siamense, Myriophyllum sibiricum, Myriophyllum simulans, Myriophyllum spicatum, Myriophyllum striatum, Myriophyllum tenellum, Myriophyllum tetrandrum, Myriophyllum trachycarpum, Myriophyllum trifidum, Myriophyllum triphyllum, Myriophyllum tuberculatum, Myriophyllum ussuriense, Myriophyllum variifolium, Myriophyllum verrucosum, Myriophyllum verticillatum, Myriophyllum votschii, Salvinia auriculata, Salvinia biloba, Salvinia cucullata, Salvinia hastata, Salvinia herzogii, Salvinia minima, Salvinia molesta, Salvinia natans, Salvinia nymphellula, Salvinia oblongifolia, Salvinia radula, Salvinia sprucei, Spirodela intermedia, Spirodela oligorrhiza, Spirodela polyrrhiza, Spirodela punctata, Spirodela sichuanensis, Wolffia angusta, Wolffia arrhiza, Wolffia australiana, Wolffia borealis, Wolffia brasiliensis, Wolffia columbiana, Wolffia cylindracea, Wolffia elongata, Wolffia globosa, Wolffia microscopica, and Wolffia neglecta, or a combination thereof.

Aspect 8: The marine biomass reactor of any one of aspects 1-7, wherein the outer shell has a cross-sectional shape selected from the group consisting of circular, rectangular, triangular, oval, square, polygonal, pentagonal, hexagonal, heptagonal, and octagonal.

Aspect 9: The marine biomass reactor of any one of aspects 1-8, wherein the water permeable outer shell material is substantially biomass impermeable.

Aspect 10: The marine biomass reactor of any one of aspects 1-9, wherein the water permeable outer shell material is a mesh.

Aspect 11: The marine biomass reactor of any one of aspects 1-10, wherein the water permeable outer shell material comprises a woven fabric or a non-woven fabric, or a combination thereof.

Aspect 12: The marine biomass reactor of any one of aspects 1-11, wherein the width of outer shell is at least 50 centimeter, 1 meter, 3 meters, 5 meters, or 10 meters.

Aspect 13: The marine biomass reactor of any one of aspects 1-11, wherein the width of outer shell is from 50 centimeter to 20 meters.

Aspect 14: The marine biomass reactor of any one of aspects 1-13, wherein the length of outer shell is at least 5 meters, 10 meters, 25 meters, 50 meters, 75 meters, 100 meters, 250 meters, 500 meters or 750 meters.

Aspect 15: The marine biomass reactor of any one of aspects 1-13, wherein the length of outer shell is from 5 meters to 1,000 meters.

Aspect 16: The marine biomass reactor of any one of aspects 1-15, wherein the spring is a wound spring.

Aspect 17: The marine biomass reactor of any one of aspects 1-16, wherein the spring extends from the first end cap to the second end cap.

Aspect 18: The marine biomass reactor of any one of aspects 1-17, wherein the first end cap, the second end cap, or both the first end cap and the second end cap are at least partially detachable.

Aspect 19: The marine biomass reactor of any one of aspects 1-18, wherein the first end cap comprises an opening configured to receive the retraction line.

Aspect 20: The marine biomass reactor of any one of aspects 1-19, wherein the outer shell is further secured to an anchor.

Aspect 21: The marine biomass reactor of any one of aspects 1-20, wherein the retraction line is further secured to an anchor.

Aspect 22: The marine biomass reactor of any one of aspects 1-21, wherein the marine biomass reactor further comprises a buoyancy device secured to the outer shell, retraction line, or both the outer shell and the retraction line.

Aspect 23: A marine biomass farm comprising two or more marine biomass reactors of any one of aspects 1-22.

Aspect 24: A method of growing a marine biomass material comprising the steps of: a) loading a marine biomass material into the inner volume of a marine biomass reactor of any one of aspects 1-22; and b) contacting the marine biomass material and at least a portion of the marine biomass reactor to water suitable for growing the marine biomass material, thereby growing the marine biomass material.

Aspect 25: The method of aspect 24, wherein the marine biomass material is allowed to move freely within the inner volume of the reactor.

Aspect 26: The method of aspects 24 or 25, wherein the marine biomass material comprises macroalgae.

Aspect 27: The method of aspects 24 or 25, wherein the marine biomass material comprises an aquatic macrophyte.

Aspect 28: The method of any one of aspects 24-27, wherein the water suitable for growing the marine biomass material is ocean water.

Aspect 29: The method of any one of aspects 24-27, wherein the water suitable for growing the marine biomass material is brackish water.

Aspect 30: The method of any one of aspects 24-29, wherein the water suitable for growing the marine biomass material is fresh water.

Aspect 31: A method of harvesting a marine biomass material comprising the steps of: a) securing the first end cap of a marine biomass reactor of any one of aspects 1-22, wherein the inner volume of the marine biomass reactor comprises a marine biomass material; b) pulling the retraction line to compress the spring, thereby compressing the outer shell relative to the central axis, thereby decreasing the length of the outer shell; c) securing the compressed outer shell, thereby preventing the outer shell from expanding in length; and d) removing at least a portion of the marine biomass material from the inner volume of the marine biomass reactor.

What is claimed is:

1. A marine biomass reactor comprising:
a) an outer shell having a central axis and a width and defining an inner volume, wherein the outer shell comprises:
i. a water permeable outer shell material;
ii. a first end cap and a second end cap spaced apart from the first end cap relative to the central axis, thereby defining a length of the outer shell; and
iii. a spring coupled to the water permeable outer shell material having a first end and a second end spaced apart relative to the central axis, wherein the spring is configured to allow the outer shell to be compressed relative to the central axis, thereby decreasing the length of the outer shell;
b) a retraction line secured to the second end cap extending within the inner volume of the outer shell towards the first end cap.

2. The marine biomass reactor of claim 1, wherein the biomass reactor further comprises a marine biomass material within the inner volume of the outer shell.

3. The marine biomass reactor of claim 2, wherein the marine biomass material comprises macroalgae.

4. The marine biomass reactor of claim 3, wherein the macroalgae is selected from the group consisting of ulva, garcilaria, and saragassum, or a combination thereof

5. The marine biomass reactor of claim 2, wherein the marine biomass material comprises an aquatic macrophyte.

6. The marine biomass reactor of claim 1, wherein the water permeable outer shell material is substantially biomass impermeable.

7. The marine biomass reactor of claim 1, wherein the water permeable outer shell material is a mesh.

8. The marine biomass reactor of claim 1, wherein the width of outer shell is at least 50 centimeter, 1 meter, 3 meters, 5 meters, or 10 meters.

9. The marine biomass reactor of claim 1, wherein the width of outer shell is from 50 centimeter to 20 meters.

10. The marine biomass reactor of claim 1, wherein the length of outer shell is at least 5 meters, 10 meters, 25 meters, 50 meters, 75 meters, 100 meters, 250 meters, 500 meters or 750 meters.

11. The marine biomass reactor of claim 1, wherein the length of outer shell is from 5 meters to 1,000 meters.

12. The marine biomass reactor of claim 1, wherein the spring is a wound spring.

13. The marine biomass reactor of claim 1, wherein the spring extends from the first end cap to the second end cap.

14. The marine biomass reactor of claim 1, wherein the first end cap comprises an opening configured to receive the retraction line.

15. The marine biomass reactor of claim 1, wherein the outer shell is further secured to an anchor.

16. The marine biomass reactor of claim 1, wherein the retraction line is further secured to an anchor.

17. The marine biomass reactor of claim 1, wherein the marine biomass reactor further comprises a buoyancy device secured to the outer shell, retraction line, or both the outer shell and the retraction line.

18. A marine biomass farm comprising two or more marine biomass reactors of clam 1.

19. A method of growing a marine biomass material comprising the steps of:
a) loading a marine biomass material into the inner volume of a marine biomass reactor of claim 1; and
b) contacting the marine biomass material and at least a portion of the marine biomass reactor to water suitable for growing the marine biomass material, thereby growing the marine biomass material.

20. A method of harvesting a marine biomass material comprising the steps of:
a) securing the first end cap of a marine biomass reactor of claim 1, wherein the inner volume of the marine biomass reactor comprises a marine biomass material;
b) pulling the retraction line to compress the spring, thereby compressing the outer shell relative to the central axis, thereby decreasing the length of the outer shell;
c) securing the compressed outer shell, thereby preventing the outer shell from expanding in length; and
d) removing at least a portion of the marine biomass material from the inner volume of the marine biomass reactor.
